(19) United States
(12) Patent Application Publication
(10) Pub. No.: US 2006/0079479 A1
(43) Pub. Date: Apr. 13, 2006

(54) Methods of inducing vasodilation

(76) Inventor: Italo Biaggioni, Nashville, TN (US)

Correspondence Address: NEEDLE & ROSENBERG, P.C., SUITE 1000, 999 PEACHTREE STREET, ATLANTA, GA, 30309-3915, US

(21) Appl. No.: 11/226,626

(22) Filed: Sep. 14, 2005

Related U.S. Application Data
(60) Provisional application No. 60/618,098, filed on Oct. 13, 2004.

(51) Int. Cl. A61K 31/7076

(52) U.S. Cl. 514/47

(57) ABSTRACT

Provided herein are methods of inducing vasodilation in a subject diagnosed with a need for vasodilation, comprising administering into a blood vessel of the subject a therapeutically effective amount of a composition consisting essentially of adenosine monophosphate in a pharmaceutically acceptable carrier, whereby the administration of the composition induces vasodilation in the subject. This abstract is intended as a scanning tool for purposes of searching in the particular art and is not intended to be limiting of the present invention.

This application claims benefit of U.S. Provisional Application No. 60/618,098, filed Oct. 13, 2004, which is hereby incorporated herein by reference in its entirety.

### ACKNOWLEDGEMENTS

This invention was made with government support under Grant NIH IROl HL67232 awarded by the National Institutes of Health. The government has certain rights in the invention.

### RELATED FIELD

This invention relates generally to methods of inducing vasodilation in a subject in need of vasodilation. Specifically, adenosine monophosphate (AMP) can be administered into a blood vessel of a subject, thereby inducing vasodilation in the subject.

### BACKGROUND

Adenosine is a substance normally present in the body and can be administered for the treatment of some heart rhythm problems and to reduce heart damage during heart attacks. In animal models, intravenous or intra-coronary artery administration of adenosine induces chemical preconditioning, thereby protecting the heart from subsequent ischemia and limiting infarct size. In humans, however, results have been contradictory and disappointing, and there are theoretical reasons why using intra-coronary artery adenosine can fail.

A problem with administering adenosine to a subject to induce vasodilation is that it is relatively quickly taken up by cells, and very little of the adenosine administered by intravenous infusion actually reaches a target tissue. Specifically, adenosine has an extremely short half-life in blood—less than 1 second—due to a very active nucleoside transporter in red blood cells. This transporter is also present in endothelial cells, and animal studies have shown that the endothelial layer of blood vessels acts as a barrier, preventing intravascular adenosine from reaching the target interstitium. Therefore, only a very small percentage of adenosine given intravenously will reach adenosine receptors on the target cells while most of it will be taken up and destroyed by red blood cells before reaching the target cells. For example, it has also been shown that interstitial concentrations of adenosine, measured with a microdialysis probe inserted in a forearm muscle of a human subject, do not increase during intra-brachial artery infusion of adenosine at doses that produced maximal forearm vasodilation.

Therefore, there remains a need for methods and compositions that achieve therapeutically effective vasodilation while avoiding the difficulties associated with administration of adenosine. A need also exists for a method for supplying an active agent to induce vasodilation, wherein the agent is not quickly metabolized, and wherein more of the agent administered by infusion reaches a target tissue. A need also exists for a method of inducing vasodilation wherein an agent more potent than adenosine can be administered in a lesser amount or in a greater concentration while still providing therapeutically effective vasodilation.

Adenosine 5′-monophosphate (AMP) is a naturally occurring compound present in every cell in the body. AMP is generally considered an intermediate metabolite of adenosine triphosphate (ATP) and is formed when ATP is used as a source of energy, such as during ischemia or increased metabolic demands. While there are specific P2 receptors for ATP and adenosine diphosphate (ADP), the immediate precursor of AMP, and specific P1 receptors for adenosine, the immediate product of AMP, there are no known cell membrane receptors for AMP. Accordingly, AMP is generally considered to be an inactive substance that works only after its conversion to an active agonist, i.e., adenosine. Therefore, one of skill would expect a comparable dose of AMP to have less potency than adenosine if this conversion is not complete, or, at most, the same potency as adenosine if the conversion is complete.

Accordingly, provided herein is a method which fulfills the aforementioned needs by inducing vasodilation in a subject diagnosed with a need for vasodilation by administering into a blood vessel of the subject a therapeutically effective amount of a composition consisting essentially of adenosine monophosphate in a pharmaceutically acceptable carrier, whereby the administration of the composition induces vasodilation in the subject

### SUMMARY

In accordance with the purpose(s) of this invention, as embodied and broadly described herein, provided herein is a method of inducing vasodilation in a subject diagnosed with a need for vasodilation, comprising administering into a blood vessel of the subject a therapeutically effective amount of a composition consisting essentially of adenosine monophosphate in a pharmaceutically acceptable carrier, whereby the administration of the composition induces vasodilation in the subject.

Also provided is a method of inducing vasodilation in a human diagnosed with a need for vasodilation, comprising administering into a blood vessel of the human a therapeutically effective amount of a composition consisting essentially of adenosine monophosphate in a pharmaceutically acceptable carrier, whereby the administration of the composition induces vasodilation in the human.

Provided is a method of inducing vasodilation in a subject diagnosed with a need for vasodilation, comprising administering into a blood vessel of the subject a therapeutically effective amount of a composition consisting essentially of adenosine monophosphate in a pharmaceutically acceptable carrier, wherein the blood vessel is not a coronary artery, whereby the administration of the composition induces vasodilation in the subject.

Also provided is a method of inducing vasodilation in a subject diagnosed with a need for vasodilation, comprising administering into a blood vessel of the subject a therapeutically effective amount of a composition consisting essentially of adenosine and adenosine monophosphate in a pharmaceutically acceptable carrier, whereby the administration of the composition induces vasodilation in the subject.

Also provided is a method of inducing vasodilation in a subject diagnosed with a need for vasodilation, comprising administering into a blood vessel of the subject a therapeutically effective amount of a composition consisting essentially of adenosine monophosphate and lidocaine in a pharmaceutically acceptable carrier, whereby the administration of the composition induces vasodilation in the subject.

Also provided is a method of diagnosing asthma in a subject, comprising inducing bronchoconstriction in the subject by administering into a blood vessel of the subject a test dose of a composition consisting essentially of adenosine monophosphate in a pharmaceutically acceptable carrier, whereby the administration of the composition induces bronchoconstriction in the subject, thereby diagnosing asthma in the subject.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the disclosed compositions and methods.

FIG. 1 shows a graph comparing the effect on forearm blood flow (FBF) of the administration of adenosine (Ado) and adenosine monophosphate (AMP). Levels were measured before (bsl), during, and after (rec) infusion of adenosine and adenosine monophosphate at 1 μmol/min.

### DETAILED DESCRIPTION

The present invention may be understood more readily by reference to the following detailed description of preferred embodiments of the invention and the Examples included therein and to the Figures and their previous and following description.

Before the present methods and compositions are disclosed and described, it is to be understood that this invention is not limited to specific methods or to specific compositions, as such may, of course, vary. It is also to be understood that the terminology used herein is for the purpose of describing particular embodiments only and is not intended to be limiting.

Disclosed are the various compounds, solvents, solutions, carriers, and/or components to be used to prepare the disclosed compositions as well as the compositions themselves to be used within the methods disclosed herein. Also disclosed are the various steps, elements, amounts, routes of administration, symptoms, and/or treatments that are used or observed when performing the disclosed methods, as well as the methods themselves. These and other materials, steps, and/or elements are disclosed herein, and it is understood that when combinations, subsets, interactions, groups, etc. of these materials are disclosed, that while specific reference of each various individual and collective combination and permutation of these compounds may not be explicitly disclosed, each is specifically contemplated and described herein.

Likewise, it is further understood that any of the various compounds, solvents, solutions, carriers, components, steps, elements, amounts, routes of administration, symptoms, treatments, or combinations or permutations thereof—whether explicitly or implicitly disclosed—are specifically contemplated and described herein and, further, can be specifically excluded from the disclosed compositions or methods.

Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, example methods and materials are now described. All publications mentioned herein are incorporated herein by reference to disclose and describe the methods and/or materials in connection with which the publications are cited.

In this specification and in the claims which follow, reference will be made to a number of terms which shall be defined to have the following meanings:

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application data are provided in a number of different formats and that these data represent endpoints and starting points and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

As used in the specification and the appended claims, “vasodilation” is synonymous with “vasodilatation” and means dilation of, or widening of, a blood vessel, especially dilation of an arteriole or artery, leading to increased blood flow. The internal diameter of the blood vessel increases, thus increasing the size of the lumen through which blood flows. Such vasodilation generally results from relaxation of the muscular wall of the vessels.

As used herein, “subject” includes, but is not limited to, a mammal (e.g., a human, horse, pig, rabbit, dog, sheep, goat, non-human primate, cow, cat, guinea pig, or rodent), a fish, a bird or a reptile or an amphibian. The term does not denote a particular age or sex. Thus, adult and newborn subjects, as well as fetuses, whether male or female, are intended to be covered. A “patient” refers to a subject afflicted with a disease or disorder. The term “patient” includes human and veterinary subjects.

As used herein, “diagnosed with a need for vasodilation” means having been subjected to a physical examination by a person of skill, for example, a physician, and found to have a condition that can be diagnosed or treated by increasing blood flow to an organ or tissue. Blood flow may be increased generally throughout a subject's body or locally increased to a particular region, for example, a particular vascular bed in an organ or tissue.

The terms “administering” and “administration” refer to methods of providing a pharmaceutical preparation to a subject. Such methods are well known to those skilled in the art and include, but are not limited to, parenteral administration, i.e., intravenous administration, intra-arterial administration, intramuscular administration, and subcutaneous administration. In particular, “administration” can be by bolus injection with a syringe and needle, or by infusion through a catheter in place within a vessel. A vessel can be an artery or a vein. Administration can be continuous or intermittent.

The term “blood vessel” includes arteries, arterioles, veins, venules, and capillaries. A blood vessel can be immediately proximate to a vascular bed. For example, an artery or arteriole is a blood vessel that is immediately proximate to and provides blood to a vascular bed.

As used herein, a “vein” is a blood vessel that carries blood from the capillaries toward the heart and also includes venules. Examples of veins include, but are not limited to, a pulmonary vein, portal vein, superior vena cava, inferior vena cava, brachial (or forearm) vein, femoral vein, and, more generally, any peripheral vein.

As used herein, an “artery” is a blood vessel that carries blood from the heart and also includes arterioles. As used herein, “artery” also includes a pulmonary artery. Examples of arteries include, but are not limited to, an aorta, brachial (or forearm) arteries, bronchial arteries, carotid arteries, cerebral arteries, ciliary arteries, coronary arteries, digital arteries, epigastric arteries, episcleral arteries, lenticulostriate arteries, meningeal arteries, mesenteric arteries, posterior cerebral arteries, renal arteries, temporal arteries, thoracic arteries, and tibial arteries.

As used herein, a “capillary” is a small blood vessel connecting an arteriole with a venule.

As used herein, a “vascular bed” is an interconnected network of blood vessels, in particular, a network of capillaries. For example, the pulmonary vascular bed describes the blood vessels of the lungs.

A “therapeutically effective amount” refers to an amount that is sufficient to achieve the desired therapeutic result or to have an effect on undesired symptoms, but is generally insufficient to cause adverse side affects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

A “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity may be maintained, for example, by the use of coating materials such as lecithin, by the maintenance of the required particle size in the case of dispersions and by the use of surfactants. These compositions may also contain adjuvants such as preservatives, wetting agents, emulsifying agents and dispersing agents. Prevention of the action of microorganisms may be ensured by the inclusion of various antibacterial and antifungal agents such as paraben, chlorobutanol, phenol, sorbic acid and the like. It may also be desirable to include isotonic agents such as sugars, sodium chloride and the like. Prolonged absorption of the injectable pharmaceutical form may be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations may be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

The present method demonstrates that AMP is more effective than adenosine in inducing vasodilation and increasing blood flow in a subject. 5′-nucleotidase, the enzyme that converts AMP into adenosine, is localized in the target membrane surface in close proximity to adenosine receptors. Accordingly, AMP is converted to adenosine at or very near the site of action, where it can activate cell receptors before it is metabolized by adenosine deaminase. By comparison, administration of adenosine itself is very inefficient because it is metabolized by the enzyme adenosine deaminase and taken up by red blood cells before it can reach and activate relevant target receptors.

It is contemplated that AMP can be used as substitute active agonist in clinical situations wherein adenosine would be indicated. Such indications include termination of supraventricular tachycardia for which adenosine is currently the drug of choice (ADENOCARD®), and induction of coronary vasodilation for the diagnosis and treatment of coronary artery disease (ADENOSCAN®). For example, AMP can be administered intravenously for the treatment of supraventricular tachycardia, for diagnostic evaluation of tachycardias, for controlled hypotension during anesthesia, and for diagnosis of neurocardiogenic syncope. Further, AMP can be administered by intravenous or intracoronary artery infusion or by intravenous or intracoronary artery bolus injection for induction of coronary vasodilation to measure coronary blood flow reserve, for diagnosis and treatment of coronary artery disease, for cardioprotection during acute coronary syndromes or in preparation of coronary interventions, and for treatment of no-reflow phenomenon. AMP can also be used to induce cardiac protection and preconditioning.

It is further contemplated that AMP can be administered intravenously to, for example, induce bronchoconstriction in a subject for the diagnosis of asthma. For example, provided is a method of diagnosing asthma in a subject, comprising inducing bronchoconstriction in the subject by administering into a blood vessel of the subject a test dose of a composition consisting essentially of adenosine monophosphate in a pharmaceutically acceptable carrier, whereby the administration of the composition induces bronchoconstriction in the subject, thereby diagnosing asthma in the subject.

Administering AMP instead of adenosine presents several advantages. For example, in addition to its greater potency, AMP is considerably more soluble than adenosine. The limit of solubility for adenosine is approximately 5 mg/ml, whereas that for AMP is approximately 400 mg/ml. Further, AMP is easy to synthesize, is readily available as a GMP product (from Clinalfa, a brand of EMD Biosciences, Inc., an Affiliate of Merck, KgaA, Darmstadt, Germany), and is very stable.

Generally, vasodilation is induced in a subject in need thereof by administering into a blood vessel of the subject a therapeutically effective amount of a composition of adenosine monophosphate in a pharmaceutically acceptable carrier. The composition can consist essentially of adenosine monophosphate in the carrier and can include other components that do not materially affect the basic and novel characteristics of the composition. In the present method, “materially affecting the basic and novel characteristics of the composition,” when administered to a subject, includes affecting vasodilation and/or increase in blood flow.

It is also contemplated that vasodilation can be induced in a subject by administering into a blood vessel of the subject a therapeutically effective amount of a composition of adenosine and adenosine monophosphate in a pharmaceutically acceptable carrier. In such a case, such a composition can consist essentially of adenosine and adenosine monophosphate in the carrier.

It is further contemplated that vasodilation can be induced in a subject by administering into a blood vessel of the subject a therapeutically effective amount of a composition of adenosine monophosphate and lidocaine in a pharmaceutically acceptable carrier. In such a case, such a composition can consist essentially of adenosine monophosphate and lidocaine in the carrier.

A subject in need of vasodilation can also require one or more ancillary procedures in order to effect the procedures disclosed herein. For example, a subject can require anesthesia or sedation before, during, and after administering AMP to induce vasodilation.

For example, in addition to a need for vasodilation, a subject can require anesthesia, analgesia, sedation, or iatrogenic hypothermia. “Anesthesia” means the loss of feeling or sensation as a result of medications or inhaled gases. Anesthesia can be general or can be limited to a localized area of a body. “Analgesia” means reduction or absence of pain in response to painful stimulation. “Sedation” means a state of reduced excitement or anxiety that is induced by the administration of a sedative agent. “latrogenic hypothermia” means decrease in the body temperature of a subject induced by a person of skill, for example, a physician. Further, a subject can require treatment for stress or pathologic hypothermia. “Stress” means mental or physical tension that results from physical, emotional, or chemical causes. “Pathologic hypothermia” means disease- or exposure-induced low core body temperature of a subject. Further, a subject can be diagnosed with other disorders known to one of skill.

Adenosine monophosphate is generally more soluble in pharmaceutically acceptable carriers than adenosine. Specifically, the solubility of adenosine is known to be approximately 5 mg/ml, whereas that for AMP is approximately 400 mg/ml. Accordingly, for example, adenosine monophosphate can be administered in a concentration of up to its solubility limit of about 400 mg/ml. As a further example, adenosine monophosphate can be administered in a concentration of from about 0.05 mg/ml to about 400 mg/ml. Accordingly, adenosine monophosphate can be administered in a concentration of 0.05, 0.1, 0.15, 0.2, 0.25, 0.3, 0.35, 0.4, 0.45, 0.5, 1, 2, 3, 5, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 mg/ml. It is also understood that any of the above-listed concentrations can be either the upper or the lower endpoint of a range of concentrations. As another example, adenosine monophosphate can be administered in a concentration of from about 0.5 mg/ml to about 50 mg/ml.

AMP can be administered by infusion, for example, to induce preconditioning or as a continuous or intermittent infusion to simulate a stress test. In such cases, generally, adenosine monophosphate can be administered by infusion to a subject in dosages similar to those used when administering adenosine. For example, adenosine monophosphate can be administered to a subject in a dosage of up to about 500 μg per kg of bodyweight per minute. As a further example, adenosine monophosphate can be administered to a subject in a dosage of from about 10 μg per kg of bodyweight per minute to about 240 μg per kg of bodyweight per minute. Accordingly, adenosine monophosphate can be administered in a dosage of 10, 20, 30, 40, 50, 60, 70, 80, 90, 100, 110, 120, 130, 140, 150, 160, 170, 180, 190, 200, 210, 220, 230, or 240 μg per kg of bodyweight per minute. It is also understood that any of the above-listed dosages can be either the upper or the lower endpoint of a range of dosages. As another example, adenosine monophosphate can be administered to a subject in a dosage of about 180 μg per kg of bodyweight per minute.

A person of skill can administer AMP at a rate similar to that used when administering adenosine. For example, the composition can be administered at a rate of up to about 25 mg/minute of AMP. Accordingly, adenosine monophosphate can be administered at a rate of 0.25, 0.5, 0.75, 1, 2, 3, 4, 5, 10, 15, 20, or 25 mg/minute of AMP. It is also understood that any of the above-listed rates can be either the upper or the lower endpoint of a range of rates. As a further example, the composition can be administered at a rate of from about 0.5 mg/minute to about 5 mg/minute of AMP. Administration of about 0.5 mg/min of AMP is approximately equivalent to 7.14 μg/kg/min for a 70 kg subject. As another example, the composition can be administered at a rate of about 3 mg/minute.

Adenosine can be administered to a subject as an intravenous bolus to, for example, terminate cardiac arrhythmias. Generally, adenosine monophosphate can be administered as a bolus as a composition in a pharmaceutically acceptable carrier in a dosage of up to, for example, about 25 mg of AMP. Accordingly, adenosine monophosphate can be administered in an intravenous bolus in a dosage of 0.25, 0.5, 0.75, 1, 2, 3, 4, 5, 6, 7, 8, 9, 10, 11, 12, 13, 14, 15, 16, 17, 18, 19, 20, 21, 22, 23, 24, or 25 mg of AMP. It is also understood that any of the above-listed dosages can be either the upper or the lower endpoint of a range of dosages. As a further example, AMP can be administered as a bolus in a dosage of from about 1 mg to about 16 mg of adenosine monophosphate. As another example, AMP can be administered as a bolus in a dosage of about 8 mg of adenosine monophosphate.

Adenosine monophosphate can also be administered as a bolus to a subject to induce vasodilation in a specific vascular bed. For example, AMP can also be given into a coronary artery as a bolus to treat the no-reflow phenomenon during angioplasty. In such administrations, AMP can be injected into a blood vessel immediately proximate to a vascular bed targeted for vasodilation. Generally, the tissue mass of a specific vascular bed varies little between subjects. Accordingly, for example, an AMP bolus is administered as a composition in a pharmaceutically acceptable carrier in a dosage of up to 500 μg of adenosine monophosphate. Accordingly, adenosine monophosphate can be administered as a bolus into a blood vessel immediately proximate to a target vascular bed in a dosage of 0.25, 0.5, 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 μg of AMP. It is also understood that any of the above-listed dosages can be either the upper or the lower endpoint of a range of dosages. For example, a bolus can be administered of from about 5 μg to about 100 μg of adenosine monophosphate. In a further example, a bolus can be administered to provide about 30 μg of adenosine monophosphate.

Wherein a composition consisting essentially of adenosine and adenosine monophosphate in a pharmaceutically acceptable carrier is administered to a subject, adenosine can be administered in a therapeutically effective amount. For example, adenosine can be administered in dosages equivalent to dosages disclosed herein for administration of adenosine monophosphate.

Wherein a composition consisting essentially of adenosine and lidocaine in a pharmaceutically acceptable carrier is administered to a subject, lidocaine can be administered in a therapeutically effective amount. For example, lidocaine can be administered to a subject in a dosage of up to about 7 mg per kg of bodyweight per minute. Accordingly, lidocaine can be administered in a dosage of 0.01, 0.025, 0.05, 0.075, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, or 7 mg per kg of bodyweight per minute. It is also understood that any of the above-listed dosages can be either the upper or the lower endpoint of a range of dosages. As a further example, lidocaine can be administered to a subject in a dosage of from about 1 mg per kg of bodyweight per minute to about 5 mg per kg of bodyweight per minute. As another example, lidocaine can be administered to a subject in a dosage of about 3 mg per kg of bodyweight per minute.

### EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

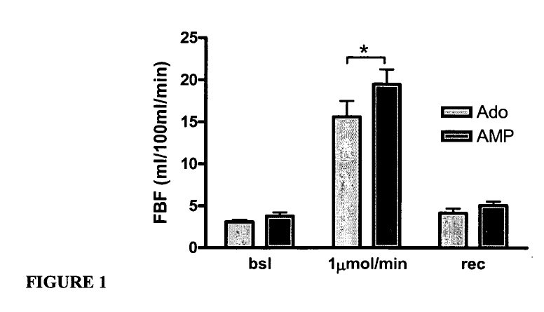

Adenosine and AMP were each separately infused into the forearm arteries of human subjects at a rate of 0.125 mg/min for 15 minutes for each dose. Blood pressure, heart rate, and respiration were continuously monitored throughout the administration. A physician was present throughout the study and monitored for side effects. At the end of each infusion period, the forearm blood flow (FBF) of each subject was then measured by standard techniques. AMP produced an increase in FBF from 3.8±0.5 to 16.3±1.6 ml/100 ml/min, whereas adenosine increased FBF from 3.1±0.2 to 14.2±2.0 ml/100 ml/min. At calculated equimolar concentrations (1 μmol/min), AMP produced significantly greater vasodilation (p<0.02) than adenosine (19.4±1.8 and 15.6±1.9 ml/100 ml/min, respectively). The results of Examples 1 and 2 are shown in FIG. 1.

### Example 2

Adenosine and AMP were each separately infused (each dose 0.5 mg/min for 15 minutes) into forearm arteries of human subjects, and the forearm blood flow (FBF) of each subject was then measured by standard techniques at the end of each infusion period. A physician was present, and the subject was monitored throughout the infusion, as in Example 1. AMP produced an increase in FBF from 3.8±0.5 to 20.7±1.8 ml/100 ml/min, whereas adenosine increased FBF from 3.1±0.2 to 17.8±1.8 m/100 ml/min. At calculated equimolar concentrations (1 μmol/min), AMP produced significantly greater vasodilation (p<0.02) than adenosine (19.4±1.8 and 15.6±1.9 ml/100 ml/min, respectively). The results of Examples 1 and 2 are shown in FIG. 1.

### Example 3

AMP can be infused into a forearm vein of a subject at a rate of 80 μg/kg/min for 15 minutes. Vasodilation and/or blood flow of the subject can then be measured by standard techniques at the end of the infusion period. It is expected that AMP would produce vasodilation and an increase in blood flow greater than that which would be produced by the infusion of an equimolar concentration of adenosine.

### Example 4

AMP can be infused into a forearm vein of a subject at a rate of 140 μg/kg/min for 15 minutes. Vasodilation and/or blood flow of the subject can then be measured by standard techniques at the end of the infusion period. It is expected that AMP would produce vasodilation and an increase in blood flow greater than that which would be produced by the infusion of an equimolar concentration of adenosine.

### Example 5

AMP can be administered by bolus injection of 8 mg of adenosine monophosphate in a pharmaceutically acceptable carrier into an antecubital or a femoral vein of a subject to, for example, terminate arrhythmia. Vasodilation and/or blood flow of the subject can then be measured by standard techniques following injection. It is expected that AMP would produce vasodilation and an increase in blood flow greater than that which would be produced by bolus injection of an equimolar concentration of adenosine. It is also contemplated that AMP can terminate arrhythmia more effectively than injection of an equimolar concentration of adenosine.

### Example 6

AMP can be administered by bolus injection of 30 μg of adenosine monophosphate in a pharmaceutically acceptable carrier into a blood vessel immediately proximate to a vascular bed targeted for vasodilation to, for example, treat the no-reflow phenomenon during angioplasty. For example, AMP can be given into a coronary artery of a subject. Vasodilation and/or blood flow of the subject can then be measured by standard techniques following injection. It is expected that AMP will produce vasodilation and an increase in blood flow greater than that which would be produced by bolus injection of an equimolar concentration of adenosine. It is also contemplated that AMP can treat the no-reflow phenomenon during angioplasty more effectively than injection of an equimolar concentration of adenosine.

### Example 7

Adenosine monophosphate in a pharmaceutically acceptable carrier can be administered by intravenous infusion into a peripheral vein of a subject to, for example, induce systemic vasodilation. For example, AMP can be given into the antecubital vein of a subject. Vasodilation and/or blood flow of the subject can then be measured by standard techniques during and following infusion. It is expected that AMP will produce systemic vasodilation and an increase in blood flow greater than that which would be produced by intravenous infusion of an equimolar amount and concentration of adenosine.

### Example 8

AMP and adenosine in a pharmaceutically-acceptable carrier can be infused into a forearm vein of a subject at a rate of 140 μg/kg/min of AMP and 80 μg/kg/min of adenosine for 15 minutes. Vasodilation and/or blood flow of the subject can then be measured by standard techniques at the end of the infusion period. It is expected that a composition of AMP and adenosine would produce vasodilation and an increase in blood flow greater than that which would be produced by the infusion of an equimolar concentration of adenosine alone.

### Example 9

AMP and lidocaine in a pharmaceutically-acceptable carrier can be infused into a forearm vein of a subject at a rate of 140 μg/kg/min of AMP and 0.05 mg/min of lidocaine for 15 minutes. Vasodilation and/or blood flow of the subject can then be measured by standard techniques at the end of the infusion period. It is expected that a composition of AMP and lidocaine would produce vasodilation and an increase in blood flow greater than that which would be produced by the infusion of an equimolar concentration of adenosine.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

Biaggioni, et al., Adenosine increases sympathetic nerve traffic in humans, 83 CIRCULATION 1668-75 (1991).
Biaggioni, et al., Cardiovascular and respiratory effects of adenosine in conscious man: Evidence for chemoreceptor activation. 61 CIRCULATION RESEARCH 779-86. (1987).
Biaggioni, et al., Cardiovascular effects of adenosine infusion in man and their modulation by dipyridamole, 39 LIFE SCIENCES 2229-36 (1986).
Boller, et al., Therapeutic action of muscle adenylic acid on ulcers and dermatitis associated with varicose orphlebitic veins; follow up report, 3 ANGIOLOGY 260-66 (1952).
Gajdos, A., A.M.P. in porphyria cutanea tarda, 1 LANCET 163 (1974).
Homeister et al., Combined Adenosine and Lidocaine Administration Limits Myocardial Reperfusion Injury, 82 CIRCULATION 595-08 (1990).
Lawrence, E. D., The use of adenylic acid suppositories in subacute thrombophlebitis and varicose ulcers. 95 AM. J. SURG. 434-37 (1958).
Lawrence, et al., Muscle adenylic acid: a clinical study of its effect, 2 ANGIOLOGY 405-11 (1951).
Lowry, et al., Adenosine-5-monophosphate in the treatment of multiple sclerosis. 226 AM. J. MED. Sci. 73-83 (1953).
Pratt, G. H., Muscle adenylic acid as an adjunct to prepare patients with phlebitis and ulcers for definitive surgery, 97 AM. J. SURG. 696-697 (1959).
Rottino, A., Effect of adenylic acid therapy upon pruritus due to Hodgkin's and other diseases, 3 CANCER 272-78 (1950).
Rottino, et al., Therapeutic action of muscle adenylic acid on ulcers and dermatitis associated with varicose orphlebitic veins; preliminary report, 1 ANGIOLOGY 194-200 (1950).
Shapiro, A., Multiple sclerosis, 147 JAMA 777-78 (1951).
Shapiro, A., Effects of muscle adenylic acid in multiple sclerosis, 58 ANN. N.Y. ACAD. SCI. 633-644 (1954).
Sherlock, C. H. & Corey, L. (1985). Adenosine monophosphate for the treatment of varicella zoster infections: a large dose of caution. JAMA. 253, 1444-1445.
Sklar, et al., Herpes zoster. The treatment and prevention of neuralgia with adenosine monophosphate, 253 JAMA 1427-30 (1985).
Sklar & Buimovici-Klein, Adenosine in the treatment of recurrent herpes labialis, 48 ORAL SURGERY, ORAL MEDICINE, ORAL PATHOLOGY 416-17 (1979).
Sklar & Wigand, Herpes zoster, 104 BRITISH JOURNAL OF DERMATOLOGY 351-52 (1981).
Steinberg, M. H., Adenosine-5-monphosphate in venous insufficiency, 9 ANGIOLOGY 154-61 (1958).

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

What is claimed is:

1. A method of inducing vasodilation in a subject diagnosed with a need for vasodilation comprising administering into a blood vessel of the subject a therapeutically effective amount of a composition consisting essentially of adenosine monophosphate in a pharmaceutically acceptable carrier, whereby the administration of the composition induces vasodilation in the subject.

2. The method of claim 1, wherein the subject is not in need of anesthesia, analgesia, sedation, or iatrogenic hypothermia.

3. The method of claim 1, wherein the subject is not in need of treatment for stress or pathologic hypothermia.

4. The method of claim 1, wherein the subject is a mammal.

5. The method of claim 4, wherein the mammal is a human.

6. The method of claim 1, wherein the blood vessel is a venule or vein.

7. The method of claim 1, wherein the blood vessel is an arteriole or artery.

8. The method of claim 7, wherein the blood vessel is immediately proximate to a vascular bed.

9. The method of claim 8, wherein adenosine monophosphate is administered in a dosage of from about 5 μg to about 100 μg.

10. The method of claim 9, wherein adenosine monophosphate is administered in a dosage of about 30 μg.

11. The method of claim 1, wherein adenosine monophosphate is administered in a concentration of from about 0.05 mg/ml to about 400 mg/ml.

12. The method of claim 11, wherein adenosine monophosphate is administered in a concentration of from about 0.5 mg/ml to about 50 mg/ml.

13. The method of claim 12, wherein adenosine monophosphate is administered in a concentration of about 10 mg/ml.

14. The method of claim 1, wherein adenosine monophosphate is administered in a dosage of from about 10 μg per kg of bodyweight per minute to about 240 μg per kg of bodyweight per minute.

15. The method of claim 14, wherein adenosine monophosphate is administered in a dosage of about 180 μg per kg of bodyweight per minute.

16. The method of claim 1, wherein adenosine monophosphate is administered in a dosage of from about 1 mg to about 16 mg.

17. The method of claim 16, wherein adenosine monophosphate is administered in a dosage of about 8 mg.

18. The method of claim 1, wherein adenosine monophosphate is administered at a rate of from about 0.5 mg/minute to about 5 mg/minute.

19. The method of claim 18, wherein adenosine monophosphate is administered at a rate of about 3 mg/minute.

20. A method of inducing vasodilation in a human diagnosed with a need for vasodilation comprising administering into a blood vessel of the human a therapeutically effective amount of a composition consisting essentially of adenosine monophosphate in a pharmaceutically acceptable carrier, whereby the administration of the composition induces vasodilation in the human.

21. A method of inducing vasodilation in a subject diagnosed with a need for vasodilation comprising administering into a blood vessel of the subject a therapeutically effective amount of a composition consisting essentially of adenosine monophosphate in a pharmaceutically acceptable carrier, wherein the blood vessel is not a coronary artery, whereby the administration of the composition induces vasodilation in the subject.

22. A method of inducing vasodilation in a subject diagnosed with a need for vasodilation comprising administering into a blood vessel of the subject a therapeutically effective amount of a composition consisting essentially of adenosine and adenosine monophosphate in a pharmaceutically acceptable carrier, whereby the administration of the composition induces vasodilation in the subject.

23. A method of inducing vasodilation in a subject diagnosed with a need for vasodilation comprising administering into a blood vessel of the subject a therapeutically effective amount of a composition consisting essentially of adenosine monophosphate and lidocaine in a pharmaceutically acceptable carrier, whereby the administration of the composition induces vasodilation in the subject.
